(19) United States
(12) Patent Application Publication
(10) Pub. No.: US 2014/0322073 A1
(43) Pub. Date: Oct. 30, 2014

(54) HAND RAIL SANITIZIER

(71) Applicants: Kurt Link, Mundelein, IL (US); William Link, Mundelein, IL (US)

(72) Inventors: Kurt Link, Mundelein, IL (US); William Link, Mundelein, IL (US)

(21) Appl. No.: 14/261,273

(22) Filed: Apr. 24, 2014

Related U.S. Application Data
(60) Provisional application No. 61/815,310, filed on Apr. 24, 2013.

(51) Int. Cl. A61L 2/10

(52) U.S. Cl. CPC A61L 2/10
USPC 422/24; 250/492.1

(57) ABSTRACT

A hand rail sanitizing system for sanitizing a hand rail of an escalator stairway from germs and bacteria on the hand rail is provided. The hand rail has a top wall, a bottom wall, and a pair of side walls. The hand rail sanitizing system comprises a container having a top wall and a bottom wall with the container positioned nearingly adjacent the hand rail. A UV-light emitting diode (LED) bulb is encased within the container. As the hand rail continuously passes by the container, the LED bulb irradiates through the bottom wall of the container toward the top wall of the hand rail.

The present application claims the benefit of priority of pending provisional patent application Ser. No. 61/815,310, filed on Apr. 24, 2013, entitled “Hand Rail Sanitizer”.

### BACKGROUND OF THE INVENTION

1. Field of the Invention

This invention relates generally to a hand rail sanitizing system and, more particularly, the invention relates to a hand rail sanitizing system for sanitizing the motorized, rubber escalator hand rails utilizing ultraviolet light to eradicate the germs and bacteria left behind on these devices.

2. Description of the Prior Art

Unfortunately, not everyone washes their hands on a regular basis and because of this, public establishments such as shopping malls and airports can be a breeding ground for germs and bacteria that cause colds, flu, and other communicable diseases. In particular, germs are commonly spread through the most basic of devices such as public telephones and escalator hand rails, the latter of which is perhaps one of the most common areas easily infiltrated by germs and bacteria. Although most people would rather not think about the potential consequences which can result from the simple action of resting the hand on the moving hand rail of an escalator, putting the hand on the surface for just a moment can result in the transfer of a slew of germs onto the skin. Picture this: a busy young mother rushes into a public restroom in order to change her baby's diaper. After changing the soiled diaper, the woman rushes out of the restroom, forgetting to wash her hands, and hurries onto the escalator. As others follow her, they are unknowingly transferring minute traces of fecal matter, urine, and bacteria left behind by the previous user, to their own, freshly washed hands. Although extremely unsettling, scenarios such as this actually occur every day across the country.

### SUMMARY

The present invention is a hand rail sanitizer, or hand rail sanitizing system for sanitizing a hand rail of an escalator stairway from germs and bacteria on the hand rail. The hand rail has a top wall, a bottom wall, and a pair of side walls. The hand rail sanitizing system comprises a container having a top wall and a bottom wall with the container positioned nearingly adjacent the hand rail. A UV-light emitting diode (LED) bulb is encased within the container. As the hand rail continuously passes by the container, the LED bulb irradiates through the bottom wall of the container toward the top wall of the hand rail.

In addition, the present invention includes a method for sanitizing a hand rail of an escalator stairway from germs and bacteria on the hand rail. The hand rail has a top wall, a bottom wall, and a pair of side walls. The method comprises providing a container having a top wall and a bottom wall, positioning the container nearingly adjacent the hand rail, encasing a UV-light emitting diode (LED) bulb within the container, continuously passing the hand rail by the container, and irradiating the LED bulb through the bottom wall of the container toward the top wall of the hand rail.

The present invention further includes a hand rail sanitizing system for sanitizing hand rails of an escalator stairway from germs and bacteria on the hand rail. The escalator stairway has a first hand rail and a second hand rail. Each hand rail has a top wall, a bottom wall, and a pair of side walls. The hand rail sanitizing system comprises a first container having a top wall and a bottom wall with the container positioned nearingly adjacent the first hand rail. A first UV-light emitting diode (LED) bulb is encased within the first container. A second container is provided having a top wall and a bottom wall. The second container is positioned nearingly adjacent the second hand rail. A second UV-light emitting diode (LED) bulb is encased within the second container. As the first hand rail continuously passes by the first container, the first LED bulb irradiates through the bottom wall of the first container toward the top wall of the first hand rail and wherein as the second hand rail continuously passes by the second container, the second LED bulb irradiates through the bottom wall of the second container toward the top wall of the second hand rail.

### BRIEF DESCRIPTION OF THE DRAWINGS

FIG. 1 is a perspective views illustrating a hand rail sanitizing system, constructed in accordance with the present invention; and

FIG. 2 is another perspective view illustrating the hand rail sanitizing system, constructed in accordance with the present invention.

### DETAILED DESCRIPTION OF THE PREFERRED EMBODIMENTS

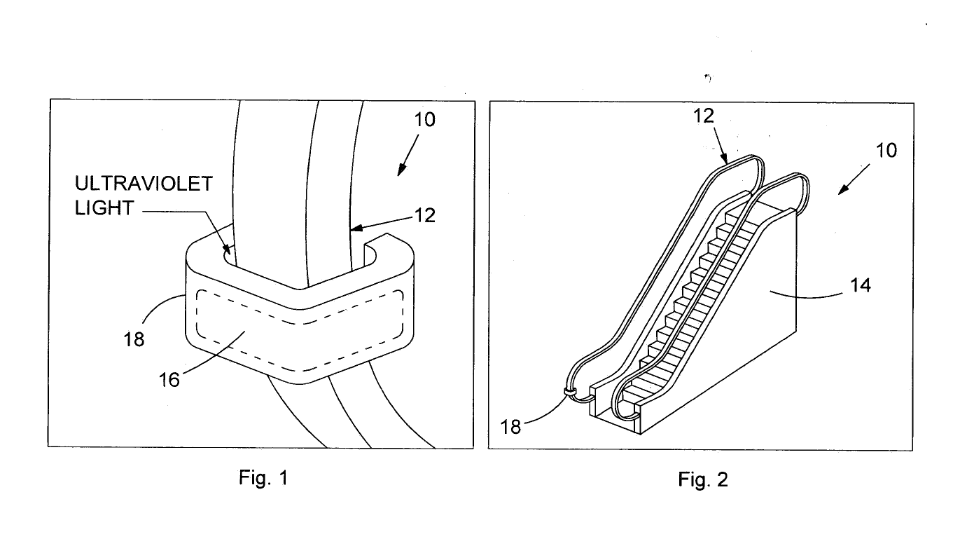

As illustrated in FIGS. 1-4, the present invention is a hand rail sanitizing system, indicated generally at 10, for sanitizing the motorized, rubber hand rails 12 of an escalator stairway 14 utilizing ultraviolet light to eradicate any germs and bacteria left behind on the hand rails 12 by previous users of the escalator stairway 14.

The hand rail sanitizing system 10 of the present invention is configured as a UV-light emitting diode (LED) bulb 16, encased within a protective box 18. In a preferred embodiment, the protective box 18 measures approximately eight inches (8″) in length, three and one half inches (3½″) in width, and four inches (4″) in depth. It should be noted that while the protective box 18 has been described as having certain dimensions, it is within the scope of the present invention for the protective box 18 to have larger dimensions or small dimensions depending on the size of the hand rail 12 and the desires of the owner. Preferably, the LED bulb 16 has a length only slightly less than the length of the protective box 18 such that the LED bulb 16 extends lengthwise within the protective box 18 substantially to each end of the protective box 18.

The LED bulb 16—protective box 18 combination is strategically positioned on the escalator stairway 14, just above the hand rail 12, so that the moving hand rail 12 continuously passes under the LED bulb 16. It is preferred to have two hand rail sanitizing systems 10 with one positioned over each hand rail 12 on the escalator stairway 18. The hand rail sanitizing system 10 can be positioned at the bottom of the escalator stairway 14, at the top of the escalator stairway 14, or in both positions, depending upon the preference of the owner. It should also be noted that the LED bulb 16—protective box 18 combination can be configured to wrap around at least the top wall and side walls of each hand rail 14 and/or can be configured to wrap around at least the top wall, the side walls, and at least a portion of the bottom wall of each hand rail 14.

Ultraviolet light has been utilized in many areas since the mid-1960s as a chemical-free means of disinfecting drinking water. The UV light penetrates a bacterial organism's cell walls and disrupts the cell's genetic material, making reproduction impossible. A special lamp generates the radiation that creates UV light by striking an electric arc through low-pressure mercury vapor. This lamp emits a broad spectrum of radiation with intense peaks at UV wavelengths of 253.7 nanometers (nm) and a lesser peak at 184.9 nm. Research has shown that the optimum UV wavelength range to destroy bacteria is between 250 nm and 270 nm. At shorter wavelengths (e.g.185 nm), UV light is powerful enough to produce ozone, hydroxyl, and other free radicals that destroy bacteria. For the hand rail sanitizing system, power can be provided by an internal battery source or via conventional 110 volts, 60 hertz power.

There are many significant benefits and advantages associated with the hand rail sanitizing system 10 of the present invention. Foremost, the hand rail sanitizing system 10 offers a simple and effective means of disinfecting the hand rails 12 on public escalator stairways 14. Cleverly incorporating an ultraviolet light source which immediately kills germs and bacteria, the installation of the hand rail sanitizing system 10 effectively eliminates the spread of germs and bacteria among motorized escalator stairway 14 users. Thwarting the spread of the many harmful agents which often thrive on hand rail 12 surfaces, the hand rail sanitizing system 10 protects the user from viruses and communicable illnesses that can potentially be transferred from one individual to the next. Users will appreciate that with the hand rail sanitizing system 10, they can safely grip any hand rail 12 on any escalator stairway 14, with the comforting knowledge that the hand rail 12 is completely free of germs and bacteria. As envisioned, the hand rail sanitizing system 10 proves particularly ideal for crowded public buildings such as airports, hotels, shopping malls, casinos, and hospitals, just a few examples of areas that experience large amounts of foot traffic. During cold and flu season, or at any time of year, the hand rail sanitizing system 10 helps keep families from catching errant maladies left behind by kids with runny noses or by travelers, shoppers, gamblers, or others who may not have washed their hands after utilizing the restroom, thus making these areas much safer for all who pass through them.

The hand rail sanitizing system 10 of the present invention provides an advanced yet simply effective means of sanitizing publicly-used escalator stairways 14. Ensuring a safe and hygienic environment for those using motorized escalator stairways 14 to traverse the floors of multilevel buildings, the sanitizer proves an invaluable commodity for any public establishment, protecting both patrons and employees.

The foregoing exemplary descriptions and the illustrative preferred embodiments of the present invention have been explained in the drawings and described in detail, with varying modifications and alternative embodiments being taught. While the invention has been so shown, described and illustrated, it should be understood by those skilled in the art that equivalent changes in form and detail may be made therein without departing from the true spirit and scope of the invention, and that the scope of the present invention is to be limited only to the claims except as precluded by the prior art. Moreover, the invention as disclosed herein may be suitably practiced in the absence of the specific elements which are disclosed herein.

What is claimed is:

1. A hand rail sanitizing system for sanitizing a hand rail of an escalator stairway from germs and bacteria on the hand rail, the hand rail having a top wall, a bottom wall, and a pair of side walls, the hand rail sanitizing system comprising:
a container having a top wall and a bottom wall, the container positioned nearingly adjacent the hand rail; and
a UV-light emitting diode (LED) bulb encased within the container;
wherein as the hand rail continuously passes by the container, the LED bulb irradiates through the bottom wall of the container toward the top wall of the hand rail.

2. The hand rail sanitizing system of claim 1 wherein a length of the LED bulb has a length less than the length of the container, the LED bulb extending lengthwise within the container substantially to each end of the container.

3. The hand rail sanitizing system of claim 1 wherein the container and encased LED bulb are positioned on the escalator stairway above the top wall of the hand rail, so that the moving hand rail continuously passes under the encased LED light.

4. The hand rail sanitizing system of claim 1 wherein the container and encased LED bulb are positioned at a bottom of the escalator stairway.

5. The hand rail sanitizing system of claim 1 wherein the container and encased LED bulb are positioned at a top of the escalator stairway.

6. The hand rail sanitizing system of claim 1 and further comprising:
an additional container;
an additional LED bulb encased within the additional container;
wherein the container and encased LED bulb are positioned at a bottom of the escalator stairway; and
wherein the additional container and additional encased LED bulb are positioned at a top of the escalator stairway.

7. The hand rail sanitizing system of claim 1 wherein the container and encased LED bulb wraps around at least the top wall and side walls of the hand rail.

8. The hand rail sanitizing system of claim 1 wherein the container and encased LED bulb wraps around at least the top wall, the side walls, and at least a portion of the bottom wall of the hand rail.

9. A method for sanitizing a hand rail of an escalator stairway from germs and bacteria on the hand rail, the hand rail having a top wall, a bottom wall, and a pair of side walls, the method comprising:
providing a container having a top wall and a bottom wall;
positioning the container nearingly adjacent the hand rail;
encasing a UV-light emitting diode (LED) bulb within the container;
continuously passing the hand rail by the container; and
irradiating the LED bulb through the bottom wall of the container toward the top wall of the hand rail.

10. A hand rail sanitizing system for sanitizing hand rails of an escalator stairway from germs and bacteria on the hand rail, the escalator stairway having a first hand rail and a second hand rail, each hand rail having a top wall, a bottom wall, and a pair of side walls, the hand rail sanitizing system comprising:
a first container having a top wall and a bottom wall, the container positioned nearingly adjacent the first hand rail;
a first UV-light emitting diode (LED) bulb encased within the first container;
a second container having a top wall and a bottom wall, the second container positioned nearingly adjacent the second hand rail; and
a second UV-light emitting diode (LED) bulb encased within the second container;
wherein as the first hand rail continuously passes by the first container, the first LED bulb irradiates through the bottom wall of the first container toward the top wall of the first hand rail; and
wherein as the second hand rail continuously passes by the second container, the second LED bulb irradiates through the bottom wall of the second container toward the top wall of the second hand rail.

11. The hand rail sanitizing system of claim 10 wherein a length of the first LED bulb has a length less than the length of the first container, the first LED bulb extending lengthwise within the first container substantially to each end of the first container and wherein a length of the second LED bulb has a length less than the length of the second container, the second LED bulb extending lengthwise within the second container substantially to each end of the second container.

12. The hand rail sanitizing system of claim 10 wherein the first container and encased first LED bulb are positioned on the escalator stairway above the top wall of the first hand rail, so that the moving first hand rail continuously passes under the encased first LED light and wherein the second container and encased second LED bulb are positioned on the escalator stairway above the top wall of the second hand rail, so that the moving second hand rail continuously passes under the encased second LED light.

13. The hand rail sanitizing system of claim 10 wherein the first container and encased first LED bulb are positioned at a bottom the first hand rail of the escalator stairway and the second container and encased second LED bulb are positioned at a bottom of the second hand rail of the escalator stairway.

14. The hand rail sanitizing system of claim 10 wherein the first container and encased first LED bulb are positioned at a top of the first hand rail of the escalator stairway and the second container and encased second LED bulb are positioned at a top of the second hand rail of the escalator stairway.

15. The hand rail sanitizing system of claim 10 wherein the first container and encased first LED bulb wraps around at least the top wall and side walls of the first hand rail and wherein the second container and encased second LED bulb wraps around at least the top wall and side walls of the second hand rail.

16. The hand rail sanitizing system of claim 10 wherein the first container and encased first LED bulb wraps around at least the top wall, the side walls, and at least a portion of the bottom wall of the first hand rail and wherein the second container and encased second LED bulb wraps around at least the top wall, the side walls, and at least a portion of the bottom wall of the second hand rail.
